(19) United States
(12) Patent Application Publication
(10) Pub. No.: US 2020/0390138 A1
(43) Pub. Date: Dec. 17, 2020

(54) DIETARY SUPPLEMENT COMPOSITIONS FOR TREATING NEUROLOGICAL DISORDERS, AND METHODS OF TREATING NEUROLOGICAL DISORDERS USING SAME

(71) Applicant: AMERICAN BLUETEC COMPANY, COMMERCE TOWNSHIP, MI (US)

(72) Inventor: Dexian DOU, COMMERCE TOWNSHIP, MI (US)

(21) Appl. No.: 16/439,201

(22) Filed: Jun. 12, 2019

(51) Int. Cl. A23L 33/105; A61K 31/05; A61P 25/28; A61K 31/714; A61K 31/122; A61K 45/06; A61K 9/00; A61P 43/00; A61K 31/315; A61K 31/10; A23L 33/15

(52) U.S. Cl. CPC A23L 33/105; A23V 2250/2132; A61P 25/28; A61K 31/714; A61K 31/122; A61K 45/06; A61K 9/0053; A61P 43/00; A61K 31/315; A61K 31/10; A23L 33/15; A23V 2002/00; A23V 2200/322; A23V 2250/314; A23V 2250/2122; A23V 2250/2104; A23V 2250/706; A23V 2250/0644; A23V 2250/21168; A61K 31/05

(57) ABSTRACT

A dietary supplement composition, which may be used for treating neurological and developmental disorders in human subjects, includes trans-resveratrol, ginkgo biloba, Coenzyme Q10, and natural flavonoids blend, in which a content of the trans-resveratrol is about 250 mg; a content of the ginkgo biloba is about 120 mg; a content of the Coenzyme Q10 is about 100 mg; and a content of the natural flavonoids blend is about 256 mg. The natural flavonoids blend may include quercetin, fisetin derived from strawberries; and anthocyanidin and anthocyanin derived from European blueberry extracts. The dietary supplement composition according another aspect includes at least three of trans-resveratrol, ginkgo biloba, Coenzyme Q10, and natural flavonoids blend. The dietary supplement compositions further includes zinc gluconate, cyanocbalamin and taurine. Such dietary supplement compositions are effective and efficient for treating subjects having neurological disorders including autism spectrum disorders, and Alzheimer's disease.

### BACKGROUND OF THE INVENTION

### 1. Field of the Invention

The present invention relates to dietary supplement compositions for treating neurological disorders, such as, autism spectrum disorders (ASD) and Alzheimer's disease and to methods of treating such disorders using the dietary supplement compositions. More particularly, the present invention relates to the use of dietary supplements and/or related adjuvant compounds to treat and/or improve the mental behavior of individuals suffering from autism, autism spectrum disorders (ASD), Alzheimer's disease and/or conditions with related symptoms such as anxiety, and methods of treatments using such compositions.

### 2. Background Art

One of concerning developmental, neurological disorders found in children is autism. Autism is also known as autism spectrum disorders (ASD). ASD is a developmental disability/disorder that causes problems with social skills and communication which can be mild, moderate or severe, and the signs and symptoms are different for every person who suffers from this disorder. Children with ASD do not follow the typical patterns of child development. Autism is a multifaceted developmental disability that interferes with the normal development of the brain in the areas of social interaction and communication skills. Another concerning neurological disorder found in older people is Alzheimer's disease which causes problems with memory, thinking and behavior in people, generally above 65 years of age.

Children with ASD have communication problems such as speaking with limited vocabulary, difficulty expressing basic wants and needs, repeating what is said, problems answering questions and problems following the directions of parents.

Further, children with ASD have memory problems such as difficulty in finding or identifying objects that are named or asked. Some children with ASD may have sleep problems.

Children with ASD also have social community problems such as poor eye contact with others, poor play skills, overly focused on a topic or objects that interest them, problems making friends, problems dealing with changes in routine, using objects in unusual ways.

In addition, children with ASD usually have no fear of real dangers. When children with ASD grow up, especially adolescent boys, they tend to become more aggressive towards and attacking parents, mostly to mothers in the worst case.

ASD affects all geographic locations. Globally, ASD knows no racial, ethnic, or social boundaries; nor family income, lifestyle, and educational levels. One distinguishing point is sex prevalence; males are affected more than the female.

Previously, ASD was thought to be rare. However, recent studies indicate that the number of ASD cases is on the rise. According to the estimates published by the National Center for Health Statistics, National Center on Birth Defects and Developmental Disabilities (see Reference 1), it has been reported that approximately 1 in 45 children aged 3-17 in the United States suffers from ASD, which is 2.22%. This disease affects the happiness of many families, but conventionally there is no single effective prescription drug to treat autism. It remains imperative to find an effective and safe method to treat and provide relief to autism patients.

Reference 1:

Zablotsky B, Black L I, Maenner M J, Schieve L A, Blumberg S J. Estimated Prevalence of Autism and Other Developmental Disabilities Following Questionnaire Changes in the 2014 National Health Interview Survey. National Health Statistics Reports 2015 Nov. 13; (87): 1-20.

ASD may develop from genetic, non-genetic, and environmental influences or the combination of all above factors. Many causes of ASD have been proposed, but understanding of the theory of causation of autism is incomplete.

The symptoms of ASD generally appear at ages 3-5 years. Clearly, it is well recognized that, autism and ASD are neurological and/or brain developmental disorders. The inventor of the present invention had long experiences of performing studies on brain development. The present inventor has been credited with discovery of brain developmental gene neuronatin (see. Reference 2).

Reference 2:

Dou D, Joseph R. Cloning of human neuronatin gene and its localization to chromosome-20q 11.2-12: the deduced protein is a novel “proteolipid”. Brain Research. 1996 Jun. 3; 723(1-2):8-22.

The present inventor also has studied for more than 20 years on neuron differentiation, aging and protection of cells, and has acquired knowledge relating to the present invention.

Currently there are no effective therapeutic agents or pharmaceutical prescription drugs available for the treatment of autism or ASD. Because there are many causes of autism, it is hard to identify a single molecule or the like to target all causes of autism.

The present invention does not focus on the causes of autism, nor does it attempt to target all causes. Although the causes for ASD are several and diverse, one thing is in common, that is: in autism spectrum disorder, the neuron cells are damaged or impaired. The present invention focuses on providing dietary supplement compositions for repairing, restoring and/or rejuvenating the damaged and impaired neurons responsible for causing ASD.

Accordingly, it is one of the objects of the present invention to provide dietary supplement compositions which are or likely to be helpful in repairing, restoring and/or rejuvenating the damaged and impaired neurons for treatment of ASD and Alzheimer's disease.

### SUMMARY OF THE INVENTION

In order to achieve the above objects, the present invention according to a first aspect thereof provides a dietary supplement composition for treating neurological disorders including ASD and Alzheimer's disease. The dietary supplement composition according to the first aspect thereof includes trans-resveratrol; ginkgo biloba; Coenzyme Q10; and natural flavonoids blend.

The present invention according to a second aspect thereof is characterized in that in the first aspect a content of the trans-resveratrol is about 250 mg; a content of the ginkgo biloba is about 120 mg; a content of the Coenzyme Q10 is about 100 mg; and a content of the natural flavonoids blend is about 256 mg.

The present invention according to a third aspect thereof in addition to the first aspect further includes at least one of zinc gluconate, cyanocbalamin and taurine.

The present invention according to a fourth aspect thereof is characterized in that in the third aspect a content of zinc gluconate is about 10 mg; a content of cyanocbalamin is about 0.25 mg and a content of taurine is about 500 mg.

The present invention according to a fifth aspect thereof in addition to the first aspect is characterized in that the natural flavonoids blend includes quercetin, fisetin derived from strawberries; and anthocyanidin and anthocyanin derived from European blueberry extracts.

The present invention according to a sixth aspect thereof in addition to the first aspect is characterized in that the composition is provided in a formulation selected from the group consisting of a capsule, a powder, a tablet, a liquid, an emulsion, a beverage and a functional food.

The present invention according to seventh aspect thereof provides a dietary supplement composition including at least three ingredients selected from the group consisting of trans-resveratrol; ginkgo biloba; Coenzyme Q10; and natural flavonoids blend.

The present invention according to an eighth aspect thereof in addition to the seventh aspect is characterized in that when present: a content of the trans-resveratrol is about 250 mg; a content of the ginkgo biloba is about 120 mg; a content of the Coenzyme Q10 is about 100 mg; and a content of the natural flavonoids blend is about 256 mg.

The present invention according to a ninth aspect thereof in addition to the eighth aspect further includes zinc gluconate, cyanocbalamin and taurine.

The present invention according to a tenth aspect thereof is characterized in that in the ninth aspect a content of the zinc gluconate is about 10 mg; a content of the cyanocbalamin is about 0.25 mg and a content of the taurine is about 500 mg.

The present invention according to an eleventh aspect in addition to the seventh aspect is characterized in that the composition is provided in a formulation selected from the group consisting of a capsule, a powder, a tablet, a liquid, an emulsion, a beverage and a functional food.

The present invention according to a twelfth aspect thereof provides a method of treating a neurological disorder in a subject. The method includes the steps of selecting a subject in need of treatment of the neurological disorder, and administering a dietary supplement composition to the subject; wherein the dietary supplement composition includes trans-resveratrol, ginkgo biloba, Coenzyme Q10, and natural flavonoids blend.

According another aspect the dietary supplement composition includes three of trans-resveratrol, ginkgo biloba, Coenzyme Q10, and natural flavonoids blend. In the dietary supplement composition, a content of the trans-resveratrol when present is about 250 mg; a content of the ginkgo biloba when present is about 120 mg; a content of the Coenzyme Q10 when present is about 100 mg; and a content of the natural flavonoids blend when present is about 256 mg. The dietary supplement composition may further include zinc gluconate, cyanocbalamin and taurine. The natural flavonoids blend includes quercetin, fisetin derived from strawberries; and anthocyanidin and anthocyanin derived from European blueberry extracts. The dietary supplement composition may be provided in a formulation selected from the group consisting of a capsule, a powder, a tablet, a liquid, an emulsion, a beverage and a functional food. The method step of administering a dietary supplement composition may include the subject ingesting an appropriate dosage the dietary supplement composition once a day over treatment period lasting several days.

The neurological disorder may be an autism spectrum disorder, an Alzhemer's disease, or any other neurological disorder such as anxiety, degenerative diseases where neurological cells are damaged or die, etc.

For a more complete understanding of the present invention, the reader is referred to the following detailed description section.

### DETAILED DESCRIPTION OF EXEMPLARY EMBODIMENTS OF THE INVENTION

Various aspects of the dietary supplement compositions for repairing, restoring and/or rejuvenating the damaged and impaired neurons in neurological disorders, e.g., for subjects of ASD and Alzheimer's disease are discussed below.

### A. Four Concepts and Aspects to Repair Damaged or Impaired Neurons

Repair of damaged neurons with autism was considered based on four concepts and aspects: (1) regeneration and remodeling, (2) improving cognitive function and memory, (3) providing energy, and (4) providing antioxidants against cell damages.

### 1. Regeneration and Remodeling

There are various dietary supplement ingredients for regeneration and remodeling of neurons.

### 1-1. Polyphenol

One of the polyphenols that may used in dietary supplement of the present invention is Trans-resveratrol (3,5,4′-trihydroxy-trans-stilbene). It is generally used as a dietary supplement. Trans-resveratrol has been used for extension of life-span of humans. It is a stilbenoid, a type of natural polyphenol found in several plants, and produced in response to injury or infections. One may get resveratrol by consuming of grapes and red wine. The resveratrol in red wine comes from the skin of grapes used to make wine.

Another polyphenol which may be used in dietary supplement of the present invention is Pterostilbene, which is an analogue of trans-resveratrol. It is a stilbenoid chemically related to resveratrol, and it serves a defensive phytoalexin role in plants.

Phlorizin (phloretin-2′-β-D-glucopyranoside) found from apple fruits, is a phytochemical that belongs to the class of polyphenols. Phlorizin is a glucoside of phloretin mainly found in the leaves of apple trees. Both of phlorizin and phloretin have bioactivity.

### 1-2. Vitamin and Amino Acids

Cyanocbalamin is a type of vitamin B12 helpful for the purpose of regeneration of neurons. Also, amino acids, L-taurine, acetyl-L-carnitine and L-tryptophan are candidates for the purpose of regeneration of neurons.

### 1-3. Fatty Acid and Membrane Stabilizer

DHA (docosahexaenoic acid), a fatty acids is a component of omega-3 fats. These fats are generally found in fish oils and seafood, with small amounts of DHA found in algae. DHA is a building block of tissue in the brain and helps with forming neural transmitters such as phosphatidylserine which is important for brain function. Children require DHA for growth and development of their bodies. The brain, central nervous system (CNS) and retina rely heavily on the adequate supply of DHA during growth.

Phosphatidylserine (PS) an essential component of healthy cell membranes, is critical to brain metabolism and function, and supports brain-cell-to-brain-cell connections. It is well recognized that 2-dimethylaminoethanol (DMAE) stabilizes cell membranes. Cell membrane degradation has been proposed as one of the prime mechanisms of dysfunction of cells. DMAE may be used in the compositions of the present invention.

### 1-4. Minerals

Mineral ions play important role in the bioprocess for regeneration and remodeling. Zinc ion is used in many reactions. Zinc gluconate is an efficient form of the supplement.

### 1-5. Peptide-Type Growth Factors

Nerve growth factor (NGF) is a neurotrophic factor and neuropeptide primarily involved in the regulation of growth, maintenance, proliferation, regeneration of neurons. Although, NGF and other peptide types of neuron growth factors may not be considered as dietary supplements, NGF and other peptide types of neuron growth factors may be considered for this purpose in the present invention.

### 2. Improving Cognitive Function and Memory

2-1. Dietary Supplements from Traditional Medicines

Ginkgo biloba, which is derived from ginkgo tree, is native to China. Most of ginkgo trees in China are over 1000 years old. It is one of oldest survived plant species. Ginkgo biloba has been effectively used by ancient Chinese in treating neurological disorders. It is believed that it may improve cognitive function and memories.

Ashwagandha, a traditional Indian ginseng, has beneficial effects on mood and helps the body to adapt to stress to promote well-being. It promotes the healthy growth of axons and dendrites, nerve cell components. Ashwagandha also inhibits an enzyme that breaks down acetylcholine, which helps to maintain cognition and memory.

### 2-2. Phospholipid

Phosphatidylcholine (PC) is a class of phospholipids that incorporate choline as a headgroup. Phosphatidylcholine exists naturally in the body in all cells. It was found in eggs, soybeans, mustard, sunflower, and other foods. The term “phosphatidylcholine” is sometimes used interchangeably with “lecithin”. Egg yolk lecithin and soybean lecithin are helpful for improving cognitive function and memories.

### 2-3. Amino Acids

Amino acids, specifically, L-phenylalanine and L-arginine are helpful for improving memory in humans. L-tryptophan is an amino acid that is important for the production of serotonin. The conditionally essential amino acid L-tyrosine is a precursor of catecholamine neurotransmitters, including dopamine. L-Theanine is helpful for increasing serotonin and dopamine. Both of serotonin and dopamine are important molecules promoting well-being and beneficial memories.

### 2-4. Vitamin B9 and Methylation

Methylation processes are important and essential for all cells in human body. Folic acid is a vitamin type of methyl group donor. Folic acid is vitamin B9 that is often generally added to processed foods and used in dietary supplements.

S-adenosyl-L-methionine (SAMe) is an universal methyl group donor in the cell. In some countries, SAMe is prescription drug, which is used for anti-depression. In United States and Europe, SAMe is a dietary supplement, SAMe is useful for the purpose of this invention. SAMe helps regulate mood and well-being.

### 2-5. Additional Ingredients

Uridine-5′-monophosphate (UMP) supports healthy brain function. UMP has been shown to promote healthy levels of neurotransmitters acetylcholine and dopamine, which supports well-being and memory.

### 3. Providing Energy

### 3-1. Molecule in the Chain of Electron Transport

Coenzyme Q10 (CoQ10) also known as ubiquinone, ubidecarenone, or coenzyme Q, is a coenzyme that is ubiquitous in animals and most bacteria, and it is necessary for the production of energy.

Nicotinamide adenine dinucleotide (NAD) is a cofactor found in all living cells. NAD, and its precursors such as niacin (NA), nicotinamide (NAM), nicotinamide riboside (NR), nicotinamide mononucleotide (NMN), are all useful for the purpose of increasing energy.

### 3-2. Energy

Adenosine triphosphate (ATP) is the direct energy for driving and functioning of many processes of brain. It is generally used as the dietary supplement.

3-3. Molecule making ATP

D-Ribose is a molecule that makes ATP.

3-4 Stimulating ATP production using traditional medicine

In Tibet, Rhodiola is a traditional medicine, which is used for the purpose of increasing mental performance, brain function. Rhodiola is also used as a dietary supplement. Rhodiola stimulates ATP production.

### 4. Antioxidants Against Cell Damages

### 4-1. Flavonoids

The general structure of flavonoids has 15-carbon skeleton, which consists of two phenyl rings and a heterocyclic ring. This unique structure enables flavonoids to exhibit antioxidant bioactivity.

Flavonoids such as quercetin, fisetin (from strawberry), anthocyanidin and anthocyanin (European blueberry extracts), are useful for the purpose to protect damaged cells with autism.

### 4-2. Carotenoid

Astaxanthin, a xanthophyll carotenoid, present in microalgae, fungi, complex plants, seafood, flamingos and quail. It is a powerful antioxidant prevents and/or minimizes damage to cells.

### 4-3. Amino Acids

Amino acids, such as N-acetyl-L-cysteine and glutathione (GSH) protect neurons. GSH, a tripeptide formed from three amino acids, is an antioxidant in plants, animals, fungi, and some bacteria and archaea.

### B. Dietary Supplement Compositions Formulations

Formula I of the dietary supplement composition of the present invention consists of following molecules selected from above “pool” of four aspects described above. In other words, the dietary supplement composition according to Formula I includes trans-resveratrol; ginkgo biloba; Coenzyme Q10; and natural flavonoids blend. Detailed amounts of each compound are provided in Example 1 herein.

### Part 1. Trans-Resveratrol

In the present formulation, trans-resveratrol may function as regeneration and remodeling. About 20 years ago, the inventor of the present invention found that trans-resveratrol could help the stem cells transforming into neuron cells. In addition, trans-resveratrol is one of the most relevant and extensively studied polyphenol. Accordingly, in the dietary supplement composition of the present invention, trans-resveratrol was chosen from the pool of molecules in the aspect “Regeneration remodeling”. Chemical structure of trans-resveratrol is illustrated in the following Illustration 1.

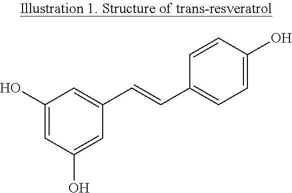 [SMILES: Oc1ccc(/C=C/c2cc(O)cc(O)c2)cc1]

Trans-resveratrol is a dietary supplement, and has been used for a long time, which proved that it is safe for human consumption. It is natural and isolated from Japanese knotweed.

However, one or more of polyphenols. such as Pterostilbene, Phlorizin, phloretin etc., may be used in combination with Trans-resveratrol or separately.

### Part 2. Ginkgo Biloba

Ginkgo biloba is the extract from the dried leaves of ginkgo tree. More specifically, the registry CAS number for ginkgo biloba is 90045-36-6 designated by the Chemical Abstracts Service (CAS). Ginkgo biloba was chosen by the present inventor as one component of the combination for the treatment of autism from the pool of possible materials mostly for the aspect of improving cognitive function and memory.

However, one or more other components for improving cognitive function and memory aspects discussed above, such as Ashwagandha, Phosphatidylcholine (PC), amino acids including L-phenylalanine and L-arginine, vitamin B9 and methylation, S-adenosyl-L-methionine (SAMe), Uridine-5′-monophosphate (UMP) may be used in combination with Ginkgo biloba or separately.

### Part 3. CoQ10

CoQ10, a known dietary supplement, has been used to improve cardiologic function. In the present invention, CoQ10 was chosen by the present inventor as one component of the combination for the treatment of autism from the pool of molecules for achieving the aspect “providing energy.” The representative chemical structure of CoQ10 used in the formulation of the present dietary supplement composition is illustrated below in Illustration 2.

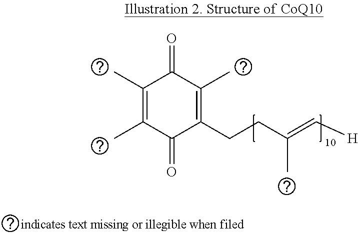

However, one or more energy providing aspects discussed above, such as NAD, NA, NAM, NR, NMN, ATP, D-Ribose, Rhodiola may be used in combination with CoQ10 or separately.

### Part 4. Flavonoids Blend

A blend of flavonoids quercetin, fisetin (from strawberry), anthocyanidin and anthocyanin (European blueberry extracts), were chosen as a part of the combination for the treatment of autism in this patent in the aspect “Antioxidants against cell damages”. Anthocyanin is anthocyanidin-glucoside. The chemical structures of quercetin, fisetin, and anthocyanidin are illustrated in Illustration 3, Illustration 4 and Illustration 5, respectively.

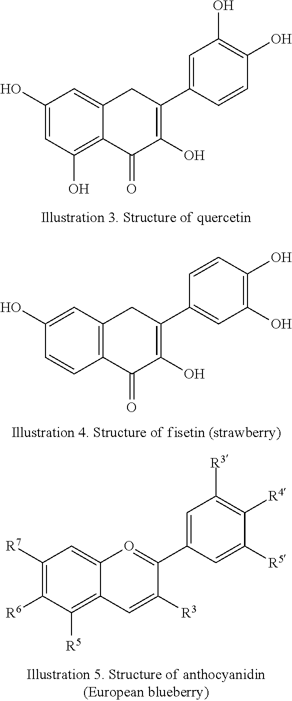

where,

### R³=OH

R⁵=OH or OCH₃

### R⁶=H or OH

R⁷=OH or OCH₃
R^{3′}H or OH or OCH₃

### R^{4′}=OH

R^{5′}=H or OH or OCH₃.

### Modifications of Dietary Supplement Composition Formulations

Formulas II through V are modifications derived from Formula I. The details of each compound is shown by Example 5.

Formula VI: Formula VI includes Formula I with addition of followings: Zinc gluconate (for example, see Illustration 6), cyanocbalamin (vitamin B12) (for example, see Illustration 7) and taurine (for example, see Illustration 8), which additional components were chosen from the pool of molecules in the aspect “Regeneration remodeling”.

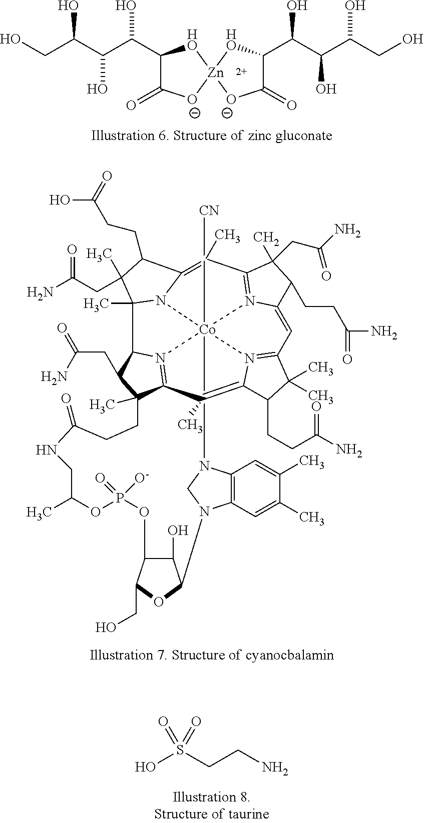

### C. Treatments Using Dietary Supplement Compositions

Before the beginning of the treatment, all children or patients with autism were diagnosed through examination by psychologists or neurologists. Parents provided data about behaviors of their children before and after the treatment, based the following categories: hyperactivity, toys destruction, playing with friends, communication with parents, verbal communication skills, aggression and learning skills. The screening methods were designed by the psychologist specifically for the purposes of this present invention.

### 1. The First Treatment of a Patient.

A male patient with autism aged 25 in New Jersey was treated with Formula I from June in 2016 (Example 2). The patient was administered Formula I once a day. The daily dosage included about 250mg trans-resveratrol, about 120 mg ginkgo biloba, about 100 mg CoQ10 and about 256 mg natural flavonoids blend providing 170 mg strawberry flavonoids (quercetin, fisetin) and about 86 mg European blueberry flavonoids(anthocyanidin, anthocyanin).

Social interest was improved in 17th week (118 days from treatment). Verbal communication skill has been dramatically improved at 19th week (133 days from treatment); he spoke a complete sentence. Previously he could only speak one word by one word. His memory has been significantly improved in 20th week (136 days from treatment). His mother told him the names of 3 items which were located at different places in a room. Then his mother walked him to other room, and asked him to get that 3 items. He did it correctly. He could only get 1 item before treatment.

Autism patients, usually have no fears of real dangers such as self-harm or death. The sentiment feeling was improved in 23th week (165 days from treatment): he cried when he heard a closed family friend passed away. Previously he never showed sadness on sad things.

One of the hard events in the life for the family with autism kids is the aggression to their parents. This patient beat on his mother several times yearly before the treatment when he got mad. After treatment in 25th week (177 days from treatment), the patient was able to completely control himself/his emotions when he got mad, and was non-violent toward his mother. His mother has never suffered from his aggression again. He is stable up to date.

Improved long term memories: Previously the patient got lost several times without parent's attention. His parents went to a shopping mall with him in 26th week (179 days from treatment), he played far away from parents when his parents were shopping, and he could return to his parents without getting lost. The learning skill has been improved in 26th week (181 days from treatment). His mother surprisingly found the patient was reading a church book in the car when his father was driving home from church. Previously he was never interested in reading a book.

The dosage was increased twice from 27th weeks, there is no side effects observed during two months.

2. A Small Trial for Treatment of Patients Aged 17-26 using Formula I.

Based on above successful test of the 25 year old male patient, a small trial of treatment was performed. A group of 37 patients with autism aged 17-26 received the treatment of Formula I (Example 3). Among them one patient dropped out of the trial during the process.

The improving rates of autism by Formula I in the study were surprisingly good, i.e., 80-94% (Table 1). To the knowledge of the present inventor, there is not any patent or publications showed an improvement efficacy rate as high as the present invention.

TABLE 1
Effect of Formula I on psychological functions and
behaviors in patients with autism aged 17-26.

| | Weeks after Treatment | Improvement (%) |
|---|---|---|
| Hyperactivity | 17 | 83 |
| Toys destruction | 17 | 89 |
| Playing with friends | 17 | 83 |
| Communication with parents | 20 | 91 |
| Verbal communication | 20 | 88 |
| Aggression | 26 | 94 |
| Learning skills | 26 | 91 |
| Sleep disorders | 26 | 86 |

There is another group of 8 patients with autism aged 17-26 treated with placebo of multivitamins. Of those that took the placebo, there was generally no improvement in any of the discussed characteristics, as only one of the eight patients reported that the communication skills improved.

### 3. A Small Trial for Treatment of Children Aged 8-16.

A group of 30 children with autism aged 8-16 were treated with Formula I, except that the dosage was half of the dosage amount of the above trail involving 37 patients (Example 4). There were 3 children who dropped out of this trial during the treatment.

Another group of 7 children with autism aged 8-16 treated with placebo multivitamins.

As shown in Table 2, the improvement rates were 91-96% for those who took the Formula I. The behaviors of children with autism were improved quicker than adult patients.

TABLE 2
Effect of treatment on psychological functions and
behaviors in children with autism aged 8-16.

| | Weeks after Treatment | Improvement (%) |
|---|---|---|
| Hyperactivity | 10 | 92 |
| Toys destruction | 10 | 88 |
| Playing with friends | 10 | 96 |
| Communication with parents | 12 | 85 |
| Verbal communication | 12 | 85 |
| Aggression | 15 | 96 |
| Learning skills | 15 | 88 |
| Sleep disorders | 15 | 81 |

There is another group of 7 children with autism aged 8-16 treated with placebo of multivitamins. No improvements were observed in any of these children.

4. The Treatment of Children Aged 8-16 with Different Formulations.

Children with autism aged 8-16 were divided into five groups, ten children in each group (Example 6). Children were treated with different formulations (Example 5).

The efficacies is 50-60% without trans-resveratrol (Formula II), 50-80% efficacies without ginkgo biloba (Formula III), 50-70% efficacies without CoQ10 (Formula IV), and 60-70% efficacies without flavonoids blend (Formula V), indicating all components are useful and necessary for obtaining high efficacies (Table 3).

Interestingly, Formula VI, Formula I adding with about 7.5mg zinc gluconate, about 0.25mg cyanocbalamin (vitamin B12) and about 250 mg taurine showed significant improvements 90-100% (Table 3). It was a small trial with 10 children, with almost 100% efficacy.

Of them, Zinc is a nutrient helping immune system and metabolism function. Zinc also is an activator of cells.

Taurine is a sulphur-containing amino acid, found in our bodies and the most animals. Taurine is often added to soda and energy drinks.

TABLE 3
Effect of treatment on psychological functions and behaviors
in children with autism aged 8-16 by different formulations.

| | | Improvement(%) | | |
|---|---|---|---|---|
| | | Verbal communication | Aggression | Learning skills |
| Formula | I | 85 | 96 | 88 |
| | II | 60 | 50 | 50 |
| | III | 50 | 70 | 80 |
| | IV | 70 | 50 | 50 |
| | V | 60 | 60 | 70 |
| | VI | 100 | 100 | 90 |

There was another group of 7 children with autism aged 8-16 treated with placebo of multivitamins. No improvements were observed in any of these patients.

### 5. The Treatment of Other Neurologic Diseases.

Six men aged 80-85 with Alzheimer's disease were treated with Formula I. The Formula I was administered once per day. The daily dosage included about 250mg trans-resveratrol, about 120mg ginkgo biloba, about 100mg CoQ10 and about 256mg natural flavonoids blend providing about 170 mg strawberry flavonoids (quercetin, fisetin) and about 86mg European blueberry flavonoids (anthocyanidin, anthocyanin).

Three of them were improved after 6 months treatments. A man fell off the bed several times a year before treatment. He hasn't fallen off again from the bed after 6 months treatment. Two of the men in this group showed improved memories. The other three men in this group have not shown any remarkable improvements.

At the same time, a control group of 4 men aged 80-85 with Alzheimer's disease were supplied with placebo multivitamins, and the men in this group showed no improvement in behaviors.

### D. EXAMPLES

### D-1. Example 1: Formula I

The dietary supplement composition according Formula I includes:

(1) about 250 mg of trans-resveratrol,
(2) about 120 mg of ginkgo biloba, (3) about 100 mg of CoQ10, and (4) about 256mg of natural flavonoids blend including quercetin, fisetin (Strawberry), anthocyanidin and anthocyanin (European blueberry extracts).

In other words, dietary supplement composition according to Formula I includes the trans-resveratrol in the amount of about 34 to 35 mass %; the ginkgo biloba in the amount of about 16 to 17 mass %; the Coenzyme Q10 in the amount of about 13 to 14 mass %; and the natural flavonoids blend in the amount of about 35 to 36 mass %. A full dosage form includes a total of about 726 mg of active ingredients including 250 mg of the trans-resveratrol, 120 mg of the ginkgo biloba, 100 mg of CoQ10, and 256 mg of the natural flavonoids blend.

It will be understood that, in view of a full dosage form of about 726 mg of active ingredients, a half dosage form includes about 363 mg of the active ingredients; and a double dosage form includes about 1452 mg of the active ingredients. A subject may be administered with a full, half or double dosage form, or a suitable dosage form based on subject's physical characteristics such as a height, a body weight, presence of other ailments, and severity of the developmental, neurological disorder.

### D-2 Example 2: Treatment of an Autistic Patient with Formula I

A male patient with autism aged 25 in New Jersey, was treated by Formula I once a day with a meal. Parents provided data about his behavior before and after the treatment each week during the first 6 months, and followed every 6 months. The daily dosage amount was increased twice beginning the 27th week. In other words, the once per day dosage for 27th week thorough 35th week was increased to double the amount the daily dosage until the 27th week.

The dosage was increased double the amounts at 27th week, and the treatment was ended at 35th week. Daily dosage from 27th to 35th week was about 500 mg trans-resveratrol, about 240 mg ginkgo biloba, about 200 mg CoQ10 and about 512 mg natural flavonoids blend providing about 340 mg strawberry flavonoids (quercetin, fisetin) and about 172 mg European blueberry flavonoids(anthocyanidin, anthocyanin).

### D-3. Example 3: A Small Trial for Treatment of Patients Aged 17-26 using Formula I

A group of 37 patients with autism aged 17-26 were treated with Formula I once a day with a meal. Another group of 8 patients with autism aged 17-26 were treated with placebo of multivitamins.

### D-4. Example 4: A Small Trial for Treatment of Kids with Autism Aged 8-16

A group of 26 kids with autism aged 8-16 were treated with Formula I. The dosage was the half of Example 1 dosage. Another group of 7 kids with autism aged 8-16 were treated with placebo multivitamins.

### D-5. Example 5: Variations of Formulations

Formula II: It is Formula I without trans-resveratrol. In other words, Formula II is a dietary supplement composition which includes ginkgo biloba, Coenzyme Q10, and natural flavonoids blend, in which a content of the ginkgo biloba is about 120 mg; a content of the Coenzyme Q10 is about 100 mg; and a content of the natural flavonoids blend is about 256 mg. It may additionally include about 10 mg of zinc gluconate, about 0.25 mg of cyanocbalamin (vitamin B12) and about 500 mg of taurine.

Formula III: It is Formula I without ginkgo biloba. In other words, Formula III is a dietary supplement composition which includes trans-resveratrol, Coenzyme Q10, and natural flavonoids blend, in which a content of the trans-resveratrol is about 250 mg; a content of the Coenzyme Q10 is about 100 mg; and a content of the natural flavonoids blend is about 256 mg. It may additionally include about 10 mg of zinc gluconate, about 0.25 mg of cyanocbalamin (vitamin B12) and about 500 mg of taurine.

Formula IV: It is Formula I without CoQ10. In other words, Formula IV is a dietary supplement composition which includes trans-resveratrol, ginkgo biloba, and natural flavonoids blend, in which a content of the trans-resveratrol is about 250 mg, a content of the ginkgo biloba is about 120 mg; and a content of the natural flavonoids blend is about 256 mg. It may additionally include about 10 mg of zinc gluconate, about 0.25 mg of cyanocbalamin (vitamin B12) and about 500 mg of taurine.

Formula V: It is Formula I without flavonoids blend. In other words, Formula V is a dietary supplement composition which includes trans-resveratrol, Coenzyme Q10 and ginkgo biloba, in which a content of the trans-resveratrol is about 250 mg, a content of the Coenzyme Q10 is about 100 mg; and a content of the ginkgo biloba is about 120 mg. It may additionally include about 10 mg of zinc gluconate, about 0.25 mg of cyanocbalamin (vitamin B12) and about 500 mg of taurine.

Formula VI: It is Formula I with addition of 10 mg zinc gluconate, 0.25 mg cyanocbalamin (vitamin B12) and 500 mg taurine.

### D-6. Example 6: The Treatment of Children Aged 8-16 using Different Formulations.

Fifty children with autism were divided to five groups, and treated with Formula II, III, IV, V and VI. The dosages were the half shown at Example 5. Another group of 7 kids with autism aged 8-16 were treated with placebo multivitamins.

### D-7. Example 7: The Treatment of Other Neurologic Diseases

Six men aged 80-85 with Alzheimer's disease were treated with Formula I. Three of six men were shown improvement after 6 months treatments. For example, one of the men who used to fall off the bed several times a year before treatment, hasn't fallen off again from the bed after 6 months treatment. Further, two of the men in this group had improved memories after the treatment. The other three men in this group have not shown remarkable improvements. Another group of 4 men aged 80-85 with Alzheimer's disease were supplied with placebo multivitamins, who showed no improvement in their behaviors.

Although the present invention has been described herein with respect to a number of specific illustrative embodiments, the foregoing description is intended to illustrate, rather than to limit the invention. Those skilled in the art will realize that many modifications of the illustrative embodiment could be made which would be operable. All such modifications, which are within the scope of the claims, are intended to be within the scope and spirit of the present invention.

What is claimed is:

1. A dietary supplement composition comprising
trans-resveratrol;
ginkgo biloba;
Coenzyme Q10; and
natural flavonoids blend.

2. A dietary supplement composition according to claim 1, wherein a content of said trans-resveratrol is about 250 mg; a content of said ginkgo biloba is about 120 mg; a content of said Coenzyme Q10 is about 100 mg; and a content of said natural flavonoids blend is about 256 mg.

3. A dietary supplement composition according to claim 1, further comprising zinc gluconate, cyanocbalamin and taurine.

4. A dietary supplement composition according to claim 3, wherein a content of zinc gluconate is about 10 mg; a content of cyanocbalamin is about 0.25 mg and a content of taurine is about 500 mg.

5. A dietary supplement composition according to claim 1, wherein said natural flavonoids blend includes quercetin, fisetin derived from strawberries; and anthocyanidin and anthocyanin derived from European blueberry extracts.

6. A dietary supplement composition according to claim 1, wherein said composition is provided in a formulation selected from the group consisting of a capsule, a powder, a tablet, a liquid, an emulsion, a beverage and a functional food.

7. A dietary supplement composition comprising at least three of:
trans-resveratrol;
ginkgo biloba;
Coenzyme Q10; and
natural flavonoids blend.

8. A dietary supplement composition according to claim 7, wherein when present: a content of said trans-resveratrol is about 250 mg; a content of said ginkgo biloba is about 120 mg;
a content of said Coenzyme Q10 is about 100 mg; and a content of said natural flavonoids blend is about 256 mg.

9. A dietary supplement composition according to claim 7, further comprising zinc gluconate, cyanocbalamin and taurine.

10. A dietary supplement composition according to claim 9, wherein a content of zinc gluconate is about 10 mg; a content of cyanocbalamin is about 0.25 mg and a content of taurine is about 500 mg.

11. A dietary supplement composition according to claim 7, wherein said composition is provided in a formulation selected from the group consisting of a capsule, a powder, a tablet, a liquid, an emulsion, a beverage and a functional food.

12. A method of treating a neurological disorder in a subject, said method comprising:
selecting a subject in need of treatment of the neurological disorder, and
administering a dietary supplement composition to said subject; said dietary supplement composition comprising trans-resveratrol, ginkgo biloba, Coenzyme Q10, and natural flavonoids blend.

13. A method of treating a neurological disorder in a subject according to claim 12, wherein said neurological disorder is an autism spectrum disorder.

14. A method of treating a neurological disorder in a subject according to claim 12, wherein said neurological disorder is an Alzhemer's disease.

15. A method of treating a neurological disorder in a subject according to claim 12, wherein in said dietary supplement composition, a content of said trans-resveratrol is about 250 mg; a content of said ginkgo biloba is about 120 mg; a content of said Coenzyme Q10 is about 100 mg; and a content of said natural flavonoids blend is about 256 mg.

16. A method of treating a neurological disorder in a subject according to claim 12, wherein said dietary supplement composition further comprises zinc gluconate, cyanocbalamin and taurine.

17. A method of treating a neurological disorder in a subject according to claim 12, wherein said natural flavonoids blend includes quercetin, fisetin derived from strawberries; and anthocyanidin and anthocyanin derived from European blueberry extracts.

18. A method of treating a neurological disorder in a subject according to claim 12, wherein said dietary supplement composition is provided in a formulation selected from the group consisting of a capsule, a powder, a tablet, a liquid, an emulsion, a beverage and a functional food.

19. A method of treating a neurological disorder in a subject according to claim 18, wherein said administering a dietary supplement composition includes said subject ingesting said dietary supplement composition once a day.
